(19) United States
(12) Patent Application Publication
(10) Pub. No.: US 2022/0009919 A1
(43) Pub. Date: Jan. 13, 2022

(54) PREPARATION OF N-MONOFLUOROALKYL COMPOUNDS

(71) Applicant: GE Healthcare Limited, Buckinghamshire (GB)

(72) Inventors: Lorenzo Williams, Oslo (NO); Gunnar Keilen, Oslo (NO); Jarle Andre Haugan, Oslo (NO)

(21) Appl. No.: 17/174,155

(22) Filed: Feb. 11, 2021

Related U.S. Application Data
(63) Continuation of application No. 15/199,108, filed on Jun. 30, 2016, now abandoned.
Application No. 15/199,108 is a continuation-in-part of application No. 13/514,074, filed on Jun. 6, 2012, now abandoned.

(30) Foreign Application Priority Data
Dec. 17, 2009 (GB) 0922023.7

(51) Int. Cl. C07D 451/02; C07F 7/00; C07F 7/22

(52) U.S. Cl. CPC C07D 451/02; C07F 7/2208; C07F 7/00

(57) ABSTRACT

The present invention relates to an improved synthesis of N-monofluoroalkyl tropanes using fluoroalkyl iodide. The invention also provides the use of such method to prepare the non-radioactive tropane intermediate FP-CIT, and its subsequent conversion to the ¹²³I-labelled radiopharmaceutical DaTSCAN™ (¹²³I-ioflupane). Also provided is the use of fluoroalkyl iodide in the alkylation method of the invention.

### CROSS-REFERENCE TO RELATED APPLICATIONS

This application is a continuation of U.S. patent application Ser. No. 15/199,108, filed Jun. 30, 2016, which is a continuation in part of U.S. patent application Ser. No. 13/514,074, filed Jun. 6, 2012, which is a filing under 35 U.S.C. § 371 of international application number PCT/EP2010/069758, filed Dec. 15, 2010, which claims priority to application number 0922023.7 file Dec. 17, 2009 in Great Britain, the entire contents of which are hereby incorporated herein by reference.

### FIELD OF THE INVENTION

The present invention relates to an improved synthesis of N-monofluoroalkyl tropanes using fluoroalkyl iodides. The invention also provides the use of such method to prepare the non-radioactive tropane intermediate FP-CIT, and its subsequent conversion to the ¹²³I-labelled radiopharmaceutical DaTSCANm (¹²³I-ioflupane).

Also provided is the use of fluoroalkyl iodides in the alkylation method of the invention.

### BACKGROUND TO THE INVENTION

DaTSCAN™ (¹²³I-ioflupane or ¹²³I FP-CIT) is prepared as follows [Neumeyer et al, J. Med. Chem., 37, 1558-1561 (1994)]:

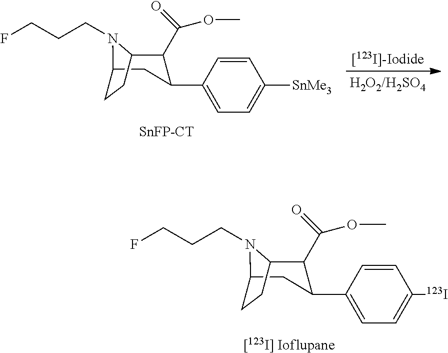

The trialkyltin non-radioactive precursor SnFP-CT is prepared from nor-beta-CIT as follows [Neumeyer et al, J. Med. Chem., 37, 1558-1561 (1994)]:

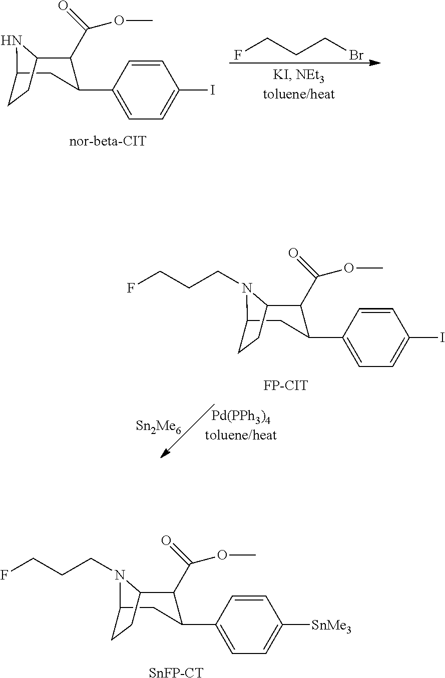

In the first step in the synthesis of SnFP-CT, the alkylation ofN-nor-beta-CIT with 1-bromo-3-fluoropropanol to yield FP-CIT, the reaction is refluxed for 9 hours and the starting material is fully consumed (>95%).

Chi et al [J. Org. Chem., 52, 658-664 (1987)] describe a method for the N-fluoroalkylation of amides and amines. The chemistry described is non-radioactive, but is designed to be suitable for adaptation to the synthesis of the corresponding ¹⁸F-labelled analogues. Spiperone was used as the model amide for amide N-alkylation, and phenylpiperazine as the model amine for amine N-alkylation. The route uses the steps:
(i) fluoride ion displacement of a haloalkyl triflate (i.e. trifluoromethanesulfonate) to give a fluoroalkyl halide;
(ii) N-alkylation of an amide/amine by the fluoroalkyl halide from step (i).

Chi et al use only fluoroalkyl bromides for amine N-alkylation.

Shiue et al [J. Lab. Comp. Radiopharm., 24, 55-64 (1987)] describe the synthesis of [¹⁸F]-fluoroalkylhalides as follows:

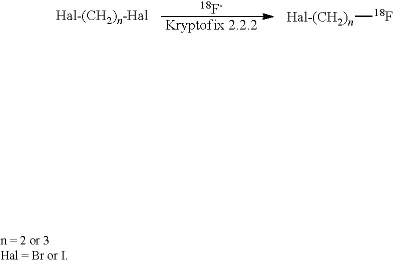

The [¹⁸F]-fluoroalkylhalides prepared were used to N-alkylate spiroperidol, normetazocine and lorazepam.

Teng et al [Nucl. Med. Biol., 17(8), 811-817 (1990)] describe the synthesis of the SCH analogues shown:

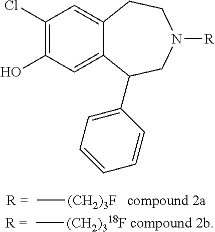

Compound 2a of Teng is the non-radioactive (¹⁹F) derivative, and is prepared by N-alkylation of the R═H compound with 1-bromo-3-fluoropropane. Compound 2b of Teng is the radioactive (¹⁸F) derivative, and is prepared by N-alkylation of the R═H compound with 3-[¹⁸F]fluoro-1-iodo-propane.

Lannoye et al [J. Med. Chem., 33, 2430-2437 (1990)] prepare N-fluoroalkyl analogues of the dopamine D-2 receptor antagonist raclopride. The alkylating agents used were 1-bromo-3-fluoropropane and 1-bromo-2-fluoroethane. Halldin et al [Nucl. Med. Biol., 18(8), 871-881 (1991)] disclose the preparation ofN-fluoroalkyl salicylamides, such as raclopride and eticlopride, having N—(CH₂)ₙ^{y}F substituents, where n is 2 or 3 and y is 18 or 19. The synthesis uses N-alkylation of the secondary amine substituent with the bromo-fluoroalkane Br—(CH₂)ₙ^{y}F.

Swahn et al [J. Lab. Comp. Radiopharm., 38, 675-685 (1996)] describe the synthesis of FP-CIT, and the corresponding N-(2-fluoroethyl) analogue (CIT-FE) via N-alkylation of nor-beta-CIT using 1-bromo-3-fluoropropane and 1-bromo-2-fluoroethane respectively.

Lundkvist et al [Nucl. Med. Biol., 24, 621-627 (1997)] prepare the ¹⁸F-labelled analogue of ¹²³I-ioflupane, where the ¹⁸F radiolabel is located in the N-fluoropropyl group, via N-alkylation of nor-beta-CIT using ¹⁸F—(CH₂)₃—Br.

Stehouwer et al [J. Med. Chem., 48, 7080-7083 (2005)] prepare N-fluoroalkyl furan-substituted tropanes as follows:

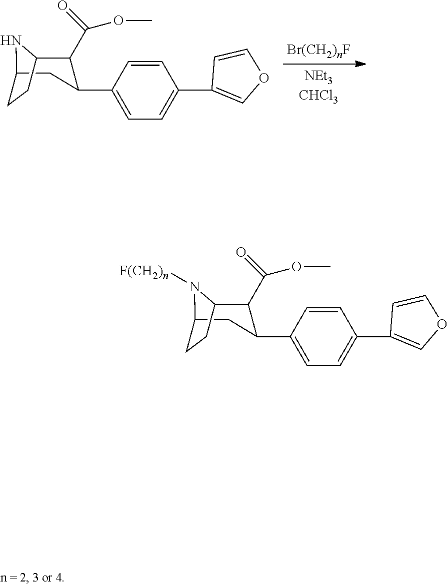

Yu et al [Bioorg. Med. Chem., 16, 6145-6155 (2008)] prepare N-fluoroalkyl isoquinoline carboxamide derivatives, via N-alkylation of a secondary amide using sodium hydride and 1-bromo-3-fluoropropane or 1-bromo-2-fluoroethane.

1-Bromo-3-fluoropropanol (BFP) is an ozone depleting agent, and is not commercially available. There is therefore a need for alternative viable syntheses of drugs and imaging agents which comprise N-fluoroalkyl substituents.

### THE PRESENT INVENTION

The conventional synthetic route to N-monofluoroalkyl tropanes employs bromo-fluoroalkanes of formula Br—(CH₂)ₙ^{y}F, where n is 2 or 3, and y is 18 or 19. There appears to be a bias in the art towards using such bromo compounds, rather than the iodo analogues. The logic is believed to be that, for simple alkylations (e.g. of sterically unhindered primary amines), there is tendency to form overalkylated products (e.g. quaternary ammonium salts) when using a reactive alkylating agent such as an iodoalkane. Thus, there is a bias toward using bromoalkanes in simple alkylations, as the lower reactivity of the bromoalkane affords improved control over the degree of alkylation.

The problem with such bromo-fluoroalkanes is that they are recognized to be ozone depleting. They are therefore increasingly less acceptable to use by the relevant regulatory authorities, and of course would not be used by environmentally-conscious drug or chemical manufacturers. Consequently, they are no longer commercially available, and their continued use is no longer appropriate. There are, however, drug and/or imaging agent products which comprise such N-monofluoroalkyl tropanes.

The alternative alkylating agent, 1-fluoro-3-iodopropane (FIP), is not ozone depleting, is commercially available, and can replace 1-bromo-3-fluoropropanol in the synthesis of FP-CIT. The present invention, which is drawn to a method of preparing N-monofluoroalkyl tropanes using FIP provides a solution to the continued manufacture of desirable N-monofluoroalkyl tropane drug and/or imaging agent products, which avoids the use of such ozone-depleting chemicals. Thus, whereas BFP is a class 1 ozone depleter with an 0.02-0.7 ODP (relative to CFC-11 which has a reference value of 1)., FIP has a boiling point of 127° C. [J. Org. Chem., 121, 748-749 (1956)], and is less volatile than BFP (boiling point 98-101° C.). The list of ozone depleting agents in the Montreal Protocol (which deals with reduced use of ozone depleting agents) includes organic molecules, usually alkyl halides, containing fluorine, chlorine or bromine. The most important process (regarding ozone depletion) is the catalytic destruction of ozone by atomic chlorine and bromine. The main source of these elements are CFC compounds (Freons) and halons (bromofluoro compounds). There are, however, no iodine-containing compounds listed—which is an advantage for the iodine-containing alkylating agents of the present invention.

The alkylating agents and methods of the present invention permit faster N-alkylation than with the bromoalkanes of the prior art. In addition, the less prone an amine is to undergo alkylation, the more likely it is that the method of the present invention will provide an improvement. In addition, the present invention also provides conditions for overcoming undesireable transesterication side reactions associated with utilizing iodine-containing alkylating agents.

### DETAILED DESCRIPTION OF THE INVENTION

In a first aspect, the present invention provides a method of preparation of an N-monofluoroalkyl tropane of Formula (IIIA):

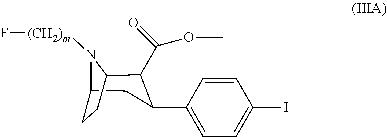

which method comprises:
(i) provision of an amine of Formula (III):

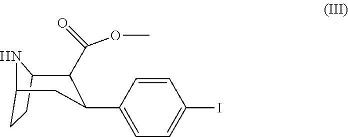 [SMILES: COC(=O)C1C2CCC(CC1c1ccc(I)cc1)N2]

(ii) alkylation of said amine with an alkylating agent of formula F—(CH₂)ₘI in the presence of a base in a suitable solvent, to give a reaction product comprising the N-monofluoroalkyl tropane of Formula (IIIA) and less than 1% of a transesterified impurity compound of formula (V):

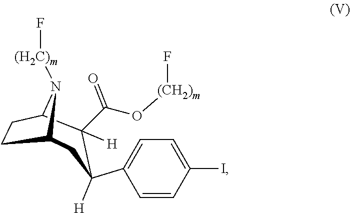

wherein m is 2, 3 or 4.

The precursor that is the amine of Formula (III) is known in the art as N-nor-beta-CIT. In one embodiment, m is 3 and Formula (IIIA) is FP-CIT or Ioflupane.

Formation of the transesterified compound (V) may be problematic at least because it is not easily removed during flash chromatographic purification of FP-CIT. However, by the methods of the present invention, formation of the trans-esterified impurity can be controlled by the experimental conditions.

In certain embodiments of the first aspect, the amount of the alkylating agent is about 1.0-1.1 equivalents, the amount of the base is about 1.0-1.1 equivalents, and the amount of the amine of Formula (III) is about 1 equivalent. In yet another embodiment, the amount of the alkylating agent is 1.1 equivalents and the amount of the base is 1.0 equivalent.

In some embodiments of the first aspect, the base is triethylamine. In some embodiments, the alkylation step includes heating in a suitable solvent. Heating is preferably at 50-120° C. in some embodiments, and in some cases may be in toluene as the preferred solvent. In yet other embodiments, it is preferred that the alkylation includes refluxing in toluene for a period of time, such as about 2 hours.

In certain embodiments of the first aspect, the reaction product includes at least 90% of the compound of Formula (IIIA). In one embodiment, the invention of the first aspect includes filtration and flash chromatographic purification of the reaction product from step (ii).

The term “base” has its conventional chemical meaning. A preferred such base is an organic base. A preferred organic basis is triethylamine.

By the term “iodide salt” is meant an ionic salt of iodide ion with an alkali metal, preferably sodium or potassium iodide, or a quaternary ammonium iodide (eg. tetrabutylammonium iodide). A preferred such salt is sodium or potassium iodide, most preferably potassium iodide.

The term “refluxing” is used as is commonly understood by one skilled in the art, wherein a reaction mixture may be heated for a period of time without solvent loss. Refluxing typically means heating a solvent or solvent mixture until the solvent(s) vaporize, and then condensing the solvent(s) to a liquid, and return to the reaction mixture, such as by using a condenser.

The term flash chromatography is as would be commonly understood by one skilled in the art, namely wherein flash chromatography is a quick, preparative column chromatography method for purifying a compound.

Suitable solvents for the alkylation reaction of step (ii) are chosen such that the precursor and alkylating agent are both soluble in the chosen solvent, and that the solvent is stable in the presence of a base. Preferred such solvents include toluene and DMF (dimethylformamide), and related solvents or mixtures thereof.

Some formulae such as the tropane of Formula (IIIA) and the precursor of Formula (III) are drawn without stereochemistry; others are drawn with stereochemistry. Nevertheless, the present formulae are intended to encompass all isomers, diastereomers and enantiomers of the chemical structures shown.

In one embodiment, the precursor of the first aspect is preferably synthetic. The term “synthetic” has the conventional meaning of the term, i.e. man-made as opposed to being isolated from natural sources eg. from the mammalian body. Such compounds have the advantage that their manufacture and impurity profile can be fully controlled.

For the alkylating agents of the first aspect in some embodiments, m is preferably 2 or 3, most preferably 3.

In the prior art alkylation of N-nor-beta-CIT, it is necessary to use potassium iodide to catalyse the alkylation using 1-bromo-3-fluoropropane, together with heating in toluene. By using 1-fluoro-3-iodopropane (FIP), the method of the first aspect is carried out in the absence of an iodide salt, which simplifies the procedure.

Nor-beta-CIT can be prepared by the method of Neumeyer et al [J. Med. Chem., 37, 1558-1561 (1994)]. It is also commercially available from ABX GmbH, Heinrich-Glaeser-Strasse 10-14 D-01454 Radeberg, Germany.

The alkylating agents of formula F—(CH₂)₃Iₘ is commercially available from Apollo Scientific (Whitefield Rd, Bredbury, Stockport, Cheshire SK6 2QR, UK) and SynQuest Laboratories, Inc. (PO Box 309, Alachua, Fl. 32616-0309, USA). It can also be prepared from the corresponding triflate F—(CH₂)₃OTf (where Tf=triflate) by the method of Chi et al [J. Org. Chem., 52, 658-664 (1987)]. The fluorinated alcohols F—(CH₂)₃OH are commercially available from Sigma-Aldrich, and can readily be converted to the corresponding sulfonate ester by standard techniques.

Several sulfonate ester alkylating agents of formula F—(CH₂)ₘOSO₂R^{a} are also commercially available. Such sulfonate esters can also be prepared by standard methods, see eg. See “March's Advanced Organic Chemistry”, fifth edition, M. B. Smith and John Wiley & Sons 2001), page 1687 which summarises such sulfonic acid ester preparation methods in the textbook.

In a second aspect, the present invention provides a method of preparation of a trialkyltin radioiodination precursor of Formula IV:

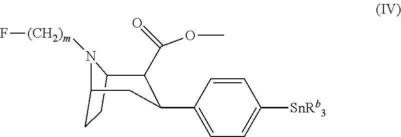

wherein said method comprises:
(i) provision of an amine of formula (III):

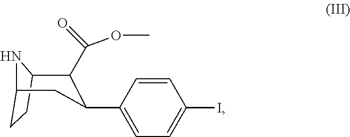 [SMILES: COC(=O)C1C2CCC(CC1c1ccc(I)cc1)N2]

(ii) alkylation of said amine with an alkylating agent of formula F—(CH₂)ₘI in the presence of a base in a suitable solvent, to give a reaction product comprising the N-monofluoroalkyl tropane of Formula (IIIA):

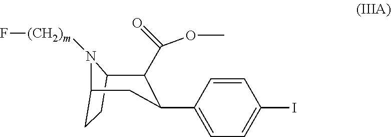

and less than 1% of a transesterified impurity compound of formula (V):

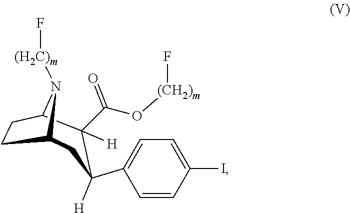

wherein m is 2, 3, or 4; and
(ii) reaction of said reaction product from step (ii) with Sn₂R^{b}₆ in the presence of a suitable catalyst to give the desired radioiodination precursor of formula (IV) wherein each R^{b} is independently C₁₋₄ alkyl, and m is 2, 3, or 4.

In one embodiment of the second aspect, m is 3. Some embodiments of the second aspect include other conditions of the first aspect, such as is in regard to amounts of reagents, solvent, etc.

For the method of the second aspect, preferred aspects thereof are as defined in the first aspect.

The organometallic precursors of Formula IV are useful in the preparation of radiopharmaceuticals, as described in the third aspect (below).

Preferred R^{b} groups are methyl and butyl, more preferably methyl. Hexamethylditin is commercially available from Sigma Aldrich. Hexabutylditin is commercially available from Merck Schuchardt & Chemos. The preparation and use of organotin precursors for radioiodination has been described by Bolton [J. Lab. Comp. Radiopharm., 45, 485-528 (2002)] and Ali et al [Synthesis, 423-445 (1996)].

In Formula (IV), wherein m is 3 and R^{b} is preferably methyl the compound of Formula IV is SnFPCT:

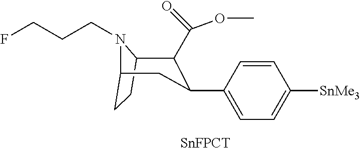

In a third aspect, the present invention provides a method of preparation of a radioiodinated tropane of Formula (IIIB):

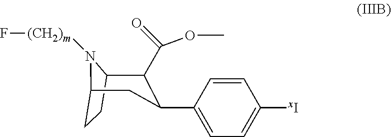

wherein said method comprises:
(i) provision of an amine of formula (III):

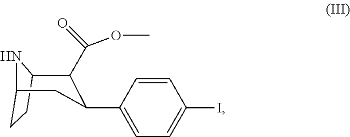 [SMILES: COC(=O)C1C2CCC(CC1c1ccc(I)cc1)N2]

(ii) alkylation of said amine with an alkylating agent of formula F—(CH₂)ₘI in the presence of a base in a suitable solvent, to give a reaction product comprising the N-monofluoroalkyl tropane of Formula (IIIA):

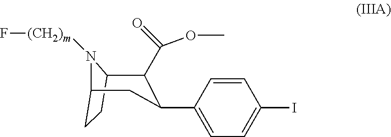

and less than 1% of a transesterified impurity compound of formula (V):

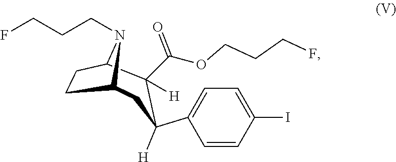

wherein m is 2, 3, or 4;
(iii) reaction of said reaction product from step (ii) with Sn₂R^{b}₆ in the presence of a suitable catalyst to give a non-radioactive trialkyltin radioiodination precursor of Formula (IV):

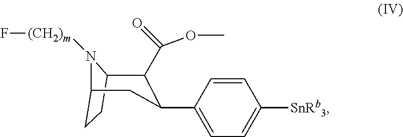

wherein m is 2, 3 or 4, and each R^{b} is independently C₁₋₄ alkyl; and
(iv) reacting the trialkyltin precursor of Formula IV from step (iii) with a supply of radioactive iodide [^{x}I]-iodide, in the presence of a suitable oxidising agent to give the desired product of Formula (IIIB) wherein where ^{x}I is ¹²⁴I or ¹²³I.

In one embodiment of Formula (IIIB), m is 3. In one embodiment, ^{x}I is preferably ¹²³I In one embodiment where m is 3 and ^{x}I is ¹²³I, the radioiodinated compound of Formula (IIIB) is ¹²³I-ioflupane.

In one exemplary embodiment, the radioiodinated tropane of Formula (IIIB) is provided as a pharmaceutical composition together with a biocompatible carrier medium. By the term “biocompatible carrier medium” is a fluid, especially a liquid, in which the labelled compound is suspended or dissolved, such that the composition is physiologically tolerable, i.e. can be administered to the mammalian body without toxicity or undue discomfort. The biocompatible carrier medium is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (eg. salts of plasma cations with biocompatible counterions), sugars (e.g. glucose or sucrose), sugar alcohols (eg. sorbitol or mannitol), glycols (eg. glycerol), or other non-ionic polyol materials (eg. polyethyleneglycols, propylene glycols and the like). The biocompatible carrier medium may also comprise biocompatible organic solvents such as ethanol. Such organic solvents are useful to solubilise more lipophilic compounds or formulations. Preferably the biocompatible carrier medium is pyrogen-free water for injection, isotonic saline or an aqueous ethanol solution. Such aqueous ethanol solutions may have a range of compositions, but 5-10% ethanol is preferred for the final composition. As indicated above, the pH of the biocompatible carrier medium for intravenous injection is suitably in the range 4.0 to 10.5. For the ¹²³I-labelled radiopharmaceuticals of the present invention, the pH of the biocompatible carrier medium is suitably 4.5 to 8.5, preferably 4.6 to 8.0, most preferably 5.0 to 7.5.

The radiopharmaceutical compositions of the present invention are suitably supplied in a clinical grade syringe or a container which is provided with a seal which is suitable for single or multiple puncturing with a hypodermic needle (e.g. a crimped-on septum seal closure) whilst maintaining sterile integrity. Such containers may contain single doses (a “unit dose”) or multiple patient doses. Suitable containers comprise a sealed vessel which permits maintenance of sterile integrity and/or radioactive safety, whilst permitting addition and withdrawal of solutions by syringe. A preferred such container is a septum-sealed vial, wherein the gas-tight closure is crimped on with an overseal (typically of aluminium). Such containers have the additional advantage that the closure can withstand vacuum if desired eg. to change the headspace gas or degas solutions.

When the radiopharmaceutical is supplied in a multiple dose container, preferred such containers comprise a single bulk vial (e.g. of 10 to 30 cm³ volume) which contains enough radiopharmaceutical for multiple patient doses. Unit patient doses can thus be withdrawn into clinical grade syringes at various time intervals during the viable lifetime of the bulk vial preparation to suit the clinical situation.

Radiopharmaceutical syringes designed to contain a single human dose, or “unit dose” and are therefore preferably a disposable or other syringe suitable for clinical use. Such syringes may optionally be provided with a syringe shield to protect the operator from radioactive dose. Suitable such radiopharmaceutical syringe shields are known in the art, and various designs are commercially available, and preferably comprise either lead or tungsten.

The radiopharmaceutical composition may optionally further comprise additional components such as an antimicrobial preservative, pH-adjusting agent or filler. By the term “antimicrobial preservative” is meant an agent which inhibits the growth of potentially harmful micro-organisms such as bacteria, yeasts or moulds. The antimicrobial preservative may also exhibit some bactericidal properties, depending on the dose. The main role of the antimicrobial preservative(s) of the present invention is to inhibit the growth of any such micro-organism in the radiopharmaceutical composition. Suitable antimicrobial preservative(s) include: the parabens, ie. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the radiopharmaceutical composition is within acceptable limits (approximately pH 4.0 to 8.5) for human or mammalian administration. Suitable such pH-adjusting agents include pharmaceutically acceptable buffers, such as tricine, phosphate buffer or TRIS [ie. tris(hydroxymethyl)aminomethane], and pharmaceutically acceptable bases such as sodium carbonate, sodium bicarbonate or mixtures thereof. For ¹²³I—FP-CIT, a preferred buffer is phosphate buffer.

By the term “filler” is meant a pharmaceutically acceptable bulking agent which may facilitate material handling during product production. Suitable fillers include inorganic salts such as sodium chloride, and water soluble sugars or sugar alcohols such as sucrose, maltose, mannitol or trehalose.

The radiopharmaceuticals of the present invention may be prepared under aseptic manufacture conditions to give the desired sterile, pyrogen-free product. The radiopharmaceuticals may also be prepared under non-sterile conditions, followed by terminal sterilisation using e.g. gamma-irradiation; autoclaving; dry heat; membrane filtration (sometimes called sterile filtration); or chemical treatment (e.g. with ethylene oxide).

In a fourth aspect, the present invention provides the use of a compound of formula F—(CH₂)ₘI as an alkylating agent in the preparation of either:
(i) the compound of Formula (IIIA) as defined in the first aspect;
(ii) the compound of Formula (IV) as defined in the second aspect; and
(iii) the compound of Formula (IIIB) as defined in the third aspect.

In the fourth aspect, the compound of formula F—(CH₂)ₘI is preferably used as an alkylating agent in the methods of preparation as described in the first, second and third aspects. Embodiments of the fourth aspect include embodiments as are described for the first, second, and third aspects.

In a fifth aspect, the present invention provides the use of an amine of Formula (III) as defined in the first aspect as a precursor in the method of preparation of the first aspect.

The invention is illustrated by the non-limiting Examples detailed below. Example 1 shows that 3-fluoro-1-iodopropane gives yields at least comparable to 1-bromo-3-fluoropropane in the N-alkylation of N-nor-R-CIT.

### Synthesis of FP-CIT Using 3-Fluoro-1-iodopropane

Example 1: N-nor-β-CIT (0.33 mmol, 123 mg) was dissolved in toluene (20 ml/gram, approx. 2 ml). Added to this solution was 3-fluoro-1-iodopropane, “FIP” (0.43 mmol, 81 mg) and triethylamine, “TEA” (0.45 mmol, 45 mg). The reaction mixture was heated to reflux under an inert atmosphere (argon/nitrogen). The reaction was essentially complete (as verified by Thin Layer Chromatography, silica TLC plates eluted with hexane-diethyl ether-triethyl amine) after 6 hours. The solvent was removed by in vacuo evaporation, and the crude was treated with diethyl ether (6 ml) and the ether phase was analysed by HPLC (reversed phase C18, gradient acetonitrile-water-phosphate buffer, UV detection at 230 nm). The crude contained 87% area of the desired FP-CIT.

Example 2: Synthesis as in Example 1 except for modification as follows: 1 equivalent N-nor-beta-CIT; 1.3 equivalents FIP and 1.35 equivalents TEA in refluxing toluene. After 5 hours reflux, the starting material was fully consumed, and work-up initiated. The purified FP-CIT contained an impurity which was identified as the trans-esterified FP-CIT, the compound of Formula (V), where m is 3.

Subsequent experiments as represented in Examples 3-5 showed that the alkylation conditions of N-nor-beta-CIT using 1-fluoro-3-iodopropane can be controlled to avoid the formation of the trans-esterified by-product, Formula (V), by lowering the excess of the alkylating agent/base, and shortening the reaction time. In some cases, this result is obtained at the expense of the yield of FP-CIT leaving starting material N-nor-beta CIT (e.g. 8%) in the reaction mixture. However, unreacted N-nor-beta-CIT is easily removed during flash chromatographic purification of FP-CIT.

Example 3: Synthesis as in Example 2, except the amount of both the alkylating agent FIP and the base TEA were 1.1 equivalents, and the time and temperature of the reaction were modified as summarized in Table 1. Progress of the reactions was monitored either by TLC or HPLC or both. Reactions were not worked-up; the results given in table 1 are from the HPLC of the crude reaction mixture.

The first experiment (FFC0064/009) was started at ambient temperature. After 21 hours, a salt (TEA*HI) had precipitated, but only modest amount of FP-CIT had been formed. The reaction temperature was raised to 50° C. and continued for a total of 28 hours. The final HPLC gave 78% FP-CIT and 19% starting material ((FFC0064/009-03). The amount of the trans-esterified impurity was 0.07%.

In another experiment (FFC0064/013) the reaction was started at 50° C. After 24 hours, TLC showed considerable amount of the starting material, and the temperature was raised to 75° C. The final HPLC analysis demonstrated 82% FP-CIT and 17% starting material and minor amounts of the trans-esterified impurity.

In another experiment (FFCO0064/015), the reaction was run at reflux, and samples analyzed each hour by HPLC. The maximum amount of FP-CIT was formed after 2 hours reflux. After three and four hours, the trans-esterified product is formed and the yield of FP-CIT was diminished.

TABLE 1

| | | | N-nor- | | FP-CIT- |
|---|---|---|---|---|---|
| | Time | Temp | beta-CIT | FP-CIT | imp. |
| Reference | (hours) | (° C.) | (% area) | (% area) | (% area) |
| FFC0064/009- | 21 | Ambient | 69 | 2 | 0 |
| FFC0064/009- | 6 | 5 | 52 | 4 | 0 |
| FFC0064/009- | 22 | 5 | 19 | 7 | 0.07 |
| FFC0064/013- | 31 | 5 | 17 | 8 | 0.5 |
| FFC0064/015- | 1 | 1 | 8 | 9 | 0.6 |
| FFC0064/015- | 2 | 1 | 8 | 9 | 0.6 |
| FFC0064/015- | 3 | 1 | 8 | 8 | 2.3 |
| FFC0064/015- | 4 | 1 | 8 | 8 | 6.1 |

Exemplary procedures for synthesis of FP-CIT according to the instant invention are provided in Examples 4 and Examples 5.

Example 4:

N-nor-beta-CIT (100 mg, 0.269 mmol) was dissolved in 2 ml toluene. 1-Fluoro-3-iodopropane (56 mg, 0.296 mmol) and triethylamine (30 mg, 0.296 mmol) were added and the reaction mixture was refluxed for 2 hours. A sample was removed and analyzed by HPLC giving FP-CIT 90%; N-nor-beta-CIT 8% and the trans-esterified FP-CIT 0.6%.

Example 5:

Dissolve N-nor-beta-CIT (1 equivalent) in toluene (19-22 ml/gram N-nor-beta-CIT). Add 1-Fluoro-3-iodopropane (1.0-1.1 equivalents) and triethylamine (1.0-1.1 equivalents) and heat the reaction mixture to reflux under an inert atmosphere. Monitor the reaction by HPLC and stop the reaction when the trans-esterified impurity starts forming (after approximately 2 hours). Work-up by filtration and flash chromatography purification (identical to the original procedure using BPP).

1-21. (canceled)

22. A method for preparing of a composition comprising a non-radioactive N-monofluoroalkyl tropane compound of Formula (IIIA): 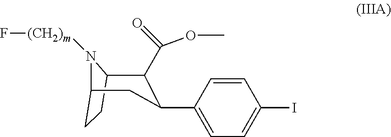
having less than 1% of a transesterified impurity compound, analyzed by HPLC, of Formula (V): 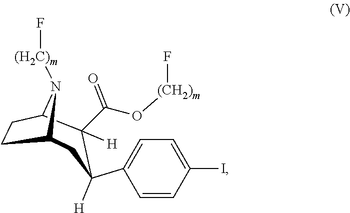
wherein m is 2, 3, or 4, the method comprising alkylation of an amine of Formula (III): 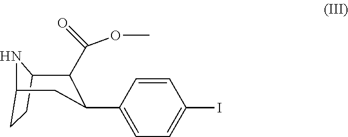 [SMILES: COC(=O)C1C2CCC(CC1c1ccc(I)cc1)N2]
with an alkylating agent of formula F—(CH₂)ₘI, in the presence of a base and a solvent.

23. The method of claim 22, wherein the alkylation comprises heating in the solvent.

24. The method of claim 22, wherein the amount of the alkylating agent is 1.0-1.1 mole equivalents, and the amount of the base is 1.0-1.1 mole equivalents.

25. The method of claim 22, wherein the heating is done at reflux for about 2 hours.

26. The method of claim 22, wherein the solvent is toluene.

27. The method of claim 22, wherein the amount of the amine of Formula (III) is 1 mole equivalent.

28. The method of claim 22, wherein the amount of the alkylating agent is 1.1 mole equivalents, and the amount of the base is 1.1 mole equivalents.

29. The method of claim 22, wherein the base is triethylamine.

30. The method of claim 22, wherein the heating is at 50-120° C.

31. The method of claim 22, wherein the alkylation comprises refluxing in the solvent.

32. The method of claim 22, wherein the composition comprises at least 90% of the compound of Formula (IIIA).

33. The method of claim 22, wherein the amount of the alkylating agent is 1.1 mole equivalents, the amount of the amine of Formula (III) is 1 mole equivalent, and the amount of the base is 1.1 mole equivalents.

34. The method of claim 22, further comprising filtration and flash chromatographic purification of the N-monofluoroalkyl tropane compound of Formula (IIIA).
